(19) United States
(12) Patent Application Publication
(10) Pub. No.: US 2025/0213866 A1
(43) Pub. Date: Jul. 3, 2025

(54) SYSTEMS WITH MULTIPLE ACTIVE IMPLANTABLE MEDICAL DEVICES

(71) Applicant: Saluda Medical Pty Limited, Macquarie Park (AU)

(72) Inventors: Peter Hubertus Aigner, Macquarie Park (AU); Jeffrey Yeu-Fai Tsang, Macquarie Park (AU); Thomas Michael Damjanovic, Macquarie Park (AU); Mohsen Nabipoor, Macquarie Park (AU); Robert Bruce Gorman, Macquarie Park (AU); Peter Scott Vallack Single, Macquarie Park (AU); Joseph Michael Flay, Macquarie Park (AU)

(21) Appl. No.: 19/002,346

(22) Filed: Dec. 26, 2024

(30) Foreign Application Priority Data
Dec. 29, 2023 (AU) 2023904255

(51) Int. Cl. A61N 1/36

(52) U.S. Cl. CPC A61N 1/36139; A61N 1/36171

(57) ABSTRACT

A multi-active-implantable-medical-device therapy system is disclosed, comprising a closed-loop neural stimulation (CLNS) device and a second active medical device. The CLNS device is configured to repeatedly execute a first activity, which involves delivering neural stimuli according to a stimulus parameter, measuring the intensities of responses evoked by the stimuli, and adjusting the stimulus parameter value to maintain the measured evoked response intensity at or near a target value. The second active medical device is configured to repeatedly execute a second activity. The system also includes a tracking device in communication with the CLNS device, which is configured to monitor a shared medium of the system to estimate the timing of the second activity. Based on the timing of the second activity, the CLNS device adjusts the timing of the first activity to mitigate interference from the second activity, thereby ensuring the effective execution of the first activity according to the adjusted timing.

The present application claims priority from Australian Provisional Patent Application No. 2023904255 filed on 29 Dec. 2023, the contents of which are incorporated herein by reference in their entirety.

### TECHNICAL FIELD

The present invention relates to active implantable devices and in particular to mitigating interference between multiple active implantable devices in a therapy system.

### BACKGROUND OF THE INVENTION

There are a range of situations in which it is desirable to apply neural stimuli in order to alter neural function, a process known as neuromodulation. For example, neuromodulation is used to treat a variety of disorders including chronic neuropathic pain, movement disorders, and pelvic floor disorders. A neuromodulation device applies an electrical pulse (stimulus) to neural tissue (fibres, or neurons) in order to generate a therapeutic effect. In general, the electrical stimulus generated by a neuromodulation device evokes a neural response known as an action potential in a neural fibre which then has either an inhibitory or excitatory effect. Inhibitory effects can be used to modulate an undesired process such as the transmission of pain, or excitatory effects may be used to cause a desired effect such as the contraction of a muscle.

When used to relieve neuropathic pain originating in the trunk and limbs, the electrical pulse is applied to the dorsal column (DC) of the spinal cord, a procedure referred to as spinal cord stimulation (SCS). Such a device typically comprises an implanted electrical pulse generator, and a power source such as a battery that may be transcutaneously rechargeable by wireless means, such as inductive transfer. An electrode array is connected to the pulse generator, and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in an orthodromic direction (in afferent fibres this means towards the head, or rostral) and in an antidromic direction (in afferent fibres this means towards the cauda, or caudal) directions. Action potentials propagating along Aβ (A-beta) fibres being stimulated in this way may inhibit the transmission of pain from a region of the body innervated by the target neural fibres (the dermatome) to the brain. To sustain the pain relief effects, stimuli are applied repeatedly, for example at a stimulus frequency in the range of 30 Hz-100 Hz.

For effective and comfortable neuromodulation, it is necessary to maintain stimulus intensity above a recruitment threshold. Stimuli below the recruitment threshold will fail to recruit sufficient neurons to generate action potentials with a therapeutic effect. In some neuromodulation applications, response from a single class of fibre is desired, but the stimulus waveforms employed can evoke action potentials in other classes of fibres which cause unwanted side effects. In pain relief, it is therefore desirable to apply stimuli with intensity below a discomfort threshold, above which uncomfortable or painful percepts arise due to over-recruitment of Aβ fibres or recruitment of undesired fibre classes. When recruitment is too large. Aβ fibres produce uncomfortable sensations. Stimulation at high intensity may even recruit Aδ (A-delta) fibres, which are sensory nerve fibres associated with acute pain, cold and heat sensation. It is therefore desirable to maintain stimulus intensity within a therapeutic range between the recruitment threshold and the discomfort threshold.

The task of maintaining appropriate neural recruitment is made more difficult by electrode migration (change in position over time) or postural changes of the implant recipient (patient), either of which can significantly alter the neural recruitment arising from a given stimulus, and therefore the therapeutic range. There is room in the epidural space for the electrode array to move, and such array movement from migration or posture change alters the electrode-to-cord distance and thus the recruitment efficacy of a given stimulus. Moreover, the spinal cord itself can move within the cerebrospinal fluid (CSF) with respect to the dura. During postural changes, the amount of CSF or the distance between the spinal cord and the electrode can change significantly. This effect is so large that postural changes alone can cause a previously comfortable and effective stimulus regime to become either ineffectual or painful.

Attempts have been made to address such problems by way of feedback or closed-loop control, such as using the methods set forth in International Patent Publication No. WO2012/155188 by the present applicant, the content of which is incorporated herein by reference. Feedback control seeks to compensate for relative nerve/electrode movement by controlling the intensity of the delivered stimuli so as to maintain neural recruitment at or near a target value. The intensity of a neural response evoked by a stimulus may be used as a feedback variable representative of the amount of neural recruitment. A signal representative of the neural response may be sensed by a measurement electrode in electrical communication with the recruited neural fibres, and processed to obtain the feedback variable. Based on the response intensity, the intensity of the applied stimulus may be adjusted to bring the response intensity closer to the target value.

It is therefore desirable to accurately measure the intensity and other characteristics of a neural response evoked by the stimulus. The action potentials generated by the depolarisation of a large number of fibres by a stimulus sum to form a measurable signal known as an evoked compound action potential (ECAP). Accordingly, an ECAP is the sum of responses from a large number of single fibre action potentials. The ECAP generated from the depolarisation of a group of similar fibres may be sensed by a measurement electrode as a positive peak potential, then a negative peak, followed by a second positive peak. This morphology is caused by the region of activation passing the measurement electrode as the action potentials propagate along the individual fibres.

Approaches proposed for obtaining a neural response measurement are described by the present applicant in International Patent Publication No. WO2012/155183, the content of which is incorporated herein by reference.

“Active” medical devices are devices that either monitor or alter the operation of some bodily process by electronic means. A closed-loop neural stimulation device is one example of an active implantable medical device (AIMD). Therapy systems involving multiple AIMDs are becoming increasingly common. For example, a patient may be prescribed with two AIMDs implanted at different locations delivering different therapies for complex conditions e.g. SCS for chronic neuropathic pain and sacral nerve stimulation for incontinence. Other examples of AIMDs include implantable cardioverter-defibrillators (ICDs), which monitor for and correct irregular heart rhythms (arrhythmias) by means of a large electrical discharge.

However, the activity of each AIMD in a multi-AIMD therapy system has the potential to interfere with the operation of another AIMD in the system. For example, if one of the devices is a closed-loop neural stimulation device that measures ECAPs during a signal window after each of its stimuli, and another neuromodulation device in the system stimulates during that window, there may be interference with the measurement that will affect the efficacy of the therapy of the closed-loop neural stimulation device. As another example, if two neuromodulation devices stimulate at the same time, their stimuli may interfere constructively or destructively with one another, affecting the efficacy of the stimuli.

In a more extreme case, the activity of one AIMD may actually damage another AIMD in the multi-AIMD therapy system. As one example, if one device is an ICD, its discharge may damage a neuromodulation device if the discharge occurs at a vulnerable time in the latter's operation cycle.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present technology as it existed before the priority date of each claim of the present disclosure.

### SUMMARY OF THE INVENTION

The present invention seeks to provide a multi-active-implantable-medical-device (AIMD) therapy system, which will overcome or substantially ameliorate at least some of the deficiencies of the prior art, or at least provide an alternative.

In particular, disclosed herein are methods for coordinating the functions of multiple AIMDs within a therapy system, and systems configured for such coordination, so as to mitigate any interference between the activities of the devices that may compromise the efficacy of the therapy. In particular, each device may explicitly coordinate with the others by means of a hand-off round in which only one device is active at a time while the others are dormant. Alternatively, each device may monitor a common medium and adjust its activity so that it may execute its activities in a way that mitigates interference by the activities of other devices.

According to a first aspect of the present technology, there is provided a multi-active-implantable-medical-device therapy system comprising: a closed-loop neural stimulation (CLNS) device configured to repeatedly execute a first activity comprising: delivering neural stimuli according to a stimulus parameter, measuring intensities of responses evoked by the stimuli, and adjusting a stimulus parameter value so as to maintain the measured evoked response intensity at or near a target value; and a second active medical device configured to repeatedly execute a second activity; and a tracking device in communication with the CLNS device, the tracking device being configured to monitor a shared medium of the system to estimate the timing of the second activity; wherein the CLNS device is configured to: adjust, based on the timing of the second activity, the timing of the first activity such that interference with the first activity by the second activity is mitigated; and execute the first activity according to the adjusted timing.

According to a second aspect of the present technology, there is provided a closed-loop neural stimulation device for controllably delivering neural stimuli, the device comprising: a stimulus source configured to provide neural stimuli to be delivered to a neural pathway of a patient in order to evoke neural responses from the neural pathway: measurement circuitry configured to capture signal windows from signals sensed on the neural pathway subsequent to respective neural stimuli; and a control unit configured to repeatedly execute a first activity comprising: controlling the stimulus source to provide a neural stimulus according to a stimulus parameter: controlling the measurement circuitry to capture a signal window subsequent to the neural stimulus: measuring an intensity of an evoked neural response in the captured signal window: determining a feedback variable from the measured intensity of the evoked neural response; and adjusting, using a feedback controller, the stimulus parameter so as to maintain the feedback variable at a target value, wherein the control unit is further configured to: monitor a medium shared with a second active medical device to estimate the timing of a second activity by the second device: adjust, based on the timing of the second activity, the timing of the first activity such that interference with the first activity by the second activity is mitigated; and execute the first activity according to the adjusted timing.

According to a third aspect of the present technology, there is provided a method of mitigating interference between devices in a multi-active-implantable-medical-device therapy system, wherein the system comprises: a closed-loop neural stimulation (CLNS) device configured to repeatedly execute a first activity comprising delivering neural stimuli according to a stimulus parameter, measuring intensities of responses evoked by the stimuli, and adjusting a stimulus parameter so as to maintain the measured evoked neural response intensity at or near a target value; and a second active medical device configured to repeatedly execute a second activity, the method comprising: monitoring a shared medium of the system to estimate the timing of the second activity: adjusting, based on the timing of the second activity, the timing of the first activity such that interference with the first activity by the second activity is mitigated; and executing the first activity according to the adjusted timing.

### BRIEF DESCRIPTION OF THE DRAWINGS

Notwithstanding any other implementations which may fall within the scope of the present invention, implementations of the invention will now be described, by way of example only, with reference to the accompanying drawings, in which:

FIG. 1 schematically illustrates an implanted neuromodulation device, according to one implementation of the present technology;

FIG. 2 is a block diagram of the neuromodulation device of FIG. 1:

FIG. 3 is a schematic illustrating interaction of the implanted neuromodulation device of FIG. 1 with a nerve;

FIG. 4 illustrates an idealised activation plot for one posture of a patient undergoing neural stimulation;

FIG. 5 is a schematic illustrating elements and inputs of a closed-loop neural stimulation (CLNS) device, according to one implementation of the present technology;

FIG. 6 is a block diagram of a multi-AIMD therapy system 600 according to one implementation of the present technology;

FIG. 7 is a flowchart illustrating a method of operating a multi-AIMD therapy system according to one aspect of the present technology;

FIG. 8 is a flowchart illustrating a method of operating a multi-AIMD therapy system according to one aspect of the present technology;

FIG. 9 is a plot illustrating interference between AIMDs in a multi-AIMD therapy system; and

FIG. 10 is a flow chart illustrating a method of operating a multi-AIMD therapy system according to one aspect of the present technology.

### DETAILED DESCRIPTION OF THE PRESENT TECHNOLOGY

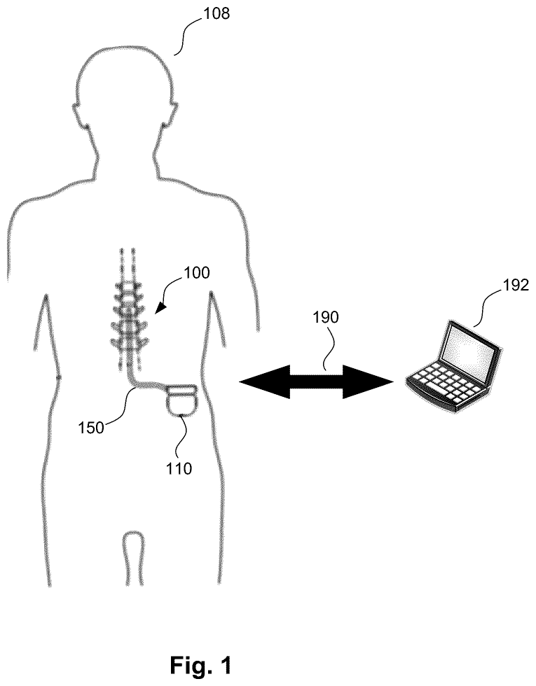

FIG. 1 schematically illustrates an implanted spinal cord neuromodulation device 100 in a patient 108, according to one implementation of the present technology. Neuromodulation device 100, which is an example of an active implantable medical device (AIMD), comprises an electronics module 110 housed within a conductive case, implanted at a suitable location. In one implementation, neuromodulation device 100 is implanted in the patient's lower abdominal area or posterior superior gluteal region. In other implementations, the electronics module 110 is implanted in other locations, such as in a flank or sub-clavicularly. The electronics module 110 is configured to electrically connect to an electrode assembly comprising an electrode array 150 implanted within the epidural space and connected to the module 110 by a suitable lead. The electrode array 150 may comprise one or more electrodes such as electrode pads on a paddle lead, circular (e.g., ring) electrodes surrounding the body of a percutaneous lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Numerous aspects of the operation of implanted neuromodulation device 100 may be programmable by an external computing device 192, which may be operable by a user such as a clinician or the patient 108. Moreover, implanted neuromodulation device 100 serves a data gathering role, with gathered data being communicated to external device 192 via a transcutaneous communications channel 190. Communications channel 190 may be active on a substantially continuous basis, at periodic intervals, at non-periodic intervals, or upon request from the external device 192. External device 192 may thus provide a clinical interface configured to program the implanted neuromodulation device 100 and recover data stored on the implanted neuromodulation device 100. This configuration is achieved by program instructions collectively referred to as the Clinical Programming Application (CPA) and stored in an instruction memory of the clinical interface.

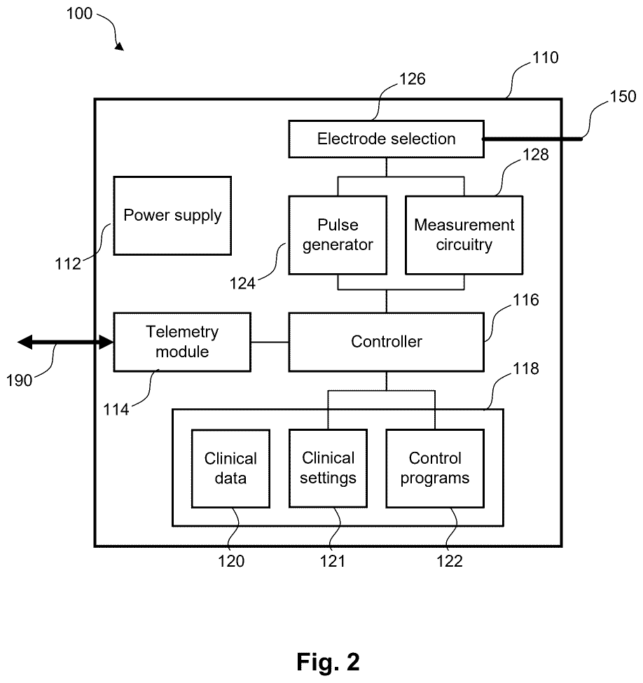

FIG. 2 is a block diagram of the neuromodulation device 100. Electronics module 110 contains a battery 112 and a telemetry module 114. In implementations of the present technology, any suitable type of transcutaneous communications channel 190, such as infrared (IR), radiofrequency (RF), capacitive or inductive transfer, may be used by telemetry module 114 to transfer power or data to and from the electronics module 110 via communications channel 190. Module controller 116 has an associated memory 118 storing one or more of clinical data 120, clinical settings 121, control programs 122, and the like. Controller 116 is configured by control programs 122, sometimes referred to as firmware, to control a pulse generator 124 to generate stimuli, such as in the form of electrical pulses, in accordance with the clinical settings 121. Electrode selection module 126 switches the generated pulses to the selected electrode(s) of electrode array 150, for delivery of the pulses to the tissue surrounding the selected electrode(s). Measurement circuitry 128, which may comprise an amplifier and an analog-to-digital converter (ADC), is configured to process signals comprising neural responses sensed by measurement electrode(s) of the electrode array 150 as selected by electrode selection module 126.

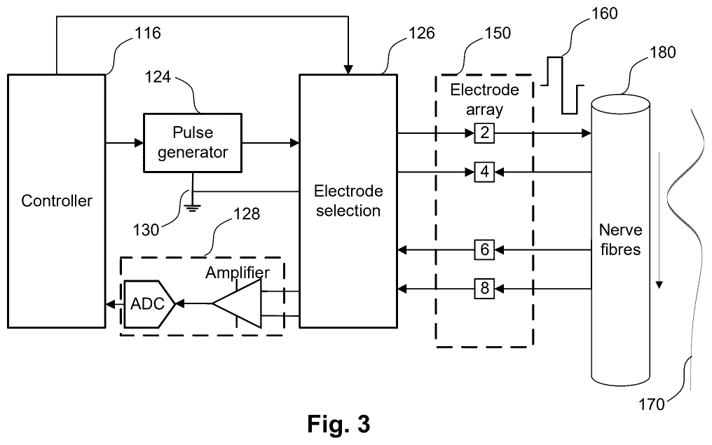

FIG. 3 is a schematic illustrating interaction of the implanted neuromodulation device 100 with a bundle of target nerve fibres 180 in the patient 108. In the implementation illustrated in FIG. 3 the target fibres 180 may be located in the spinal cord, however in alternative implementations the neuromodulation device 100 may be positioned adjacent any target neural tissue including a peripheral nerve, visceral nerve, sacral nerve, parasympathetic nerve or a brain structure. Electrode selection module 126 selects a stimulus electrode 2 of electrode array 150 through which to deliver a pulse from the pulse generator 124 to surrounding neural tissue including target fibres 180. A pulse may comprise one or more phases. e.g. a monophasic pulse comprises one phase, and a biphasic stimulus pulse 160 comprises two phases. Electrode selection module 126 also selects a return electrode 4 of the electrode array 150 for stimulus current return in each phase, to maintain a zero net charge transfer. An electrode may act as both a stimulus electrode and a return electrode over a complete multiphasic stimulus pulse. The use of two electrodes in this manner for delivering and returning current in each stimulus phase is referred to as bipolar stimulation. Alternative implementations may apply other forms of bipolar stimulation, or may use a greater number of stimulus or return electrodes. By contrast, in monopolar stimulation, current is returned through the conductive case of the neuromodulation device 100, which may therefore be configured and function as an electrode though it is not physically part of the electrode array 150. The set of stimulus electrodes and return electrodes is referred to as the stimulus electrode configuration. Electrode selection module 126 is illustrated as connecting to a ground 130 of the pulse generator 124 to enable stimulus current return via the return electrode 4. However, other connections for current return may be used in other implementations.

Delivery of an appropriate stimulus via electrodes 2 and 4 to the target fibres 180 evokes a neural response 170 comprising an evoked compound action potential (ECAP) which will propagate along the target fibres 180 as illustrated at a rate known as the conduction velocity. The ECAP may be evoked for therapeutic purposes, which in the case of spinal cord stimulation for chronic pain may be to create paresthesia at a desired location. To this end, the electrodes 2 and 4 are used to deliver stimuli periodically at any therapeutically suitable stimulus frequency, for example 30 Hz, although other frequencies may be used including frequencies as high as the kHz range. In alternative implementations, stimuli may be delivered in a non-periodic manner such as in bursts, or sporadically, as appropriate for the patient 108. To program the neuromodulation device 100 to the patient 108, a user may cause the neuromodulation device 100 to deliver stimuli of various configurations which seek to produce a sensation that may be experienced by the patient as paresthesia. When a stimulus electrode configuration is found which evokes paresthesia in a location and of a size which is congruent with the area of the patient's body affected by pain and of a quality that is comfortable for the patient, the user nominates that configuration for ongoing use. The therapy parameters may be loaded into the memory 118 of the neuromodulation device 100 as the clinical settings 121.

The neuromodulation device 100 is further configured to measure the intensity of ECAPs 170 propagating along target fibres 180, whether such ECAPs are evoked by the stimulus from electrodes 2 and 4, or otherwise evoked. To this end, any electrodes of the array 150 may be selected by the electrode selection module 126 to serve as recording electrode 6 and reference electrode 8, whereby the electrode selection module 126 selectively connects the chosen electrodes to the inputs of the measurement circuitry 128. Thus, signals sensed by the measurement electrodes 6 and 8 subsequent to the respective stimuli are passed to the measurement circuitry 128, which may comprise a differential amplifier and an analog-to-digital converter (ADC), as illustrated in FIG. 3. The recording electrode and the reference electrode are referred to as the measurement electrode configuration. The measurement circuitry 128 for example may operate in accordance with the teachings of the above-mentioned International Patent Publication No. WO2012/155183.

Signals sensed by the measurement electrodes 6, 8 and processed by measurement circuitry 128 are further processed by an ECAP detector implemented within controller 116, configured by control programs 122, to obtain information regarding the effect of the applied stimulus upon the target fibres 180. In some implementations, the sensed signals are processed by the ECAP detector in a manner which measures and stores one or more characteristics from each evoked neural response or group of evoked neural responses contained in the sensed signal. In one such implementation, the characteristics comprise a peak-to-peak ECAP amplitude in microvolts (μV). For example, the sensed signals may be processed by the ECAP detector to determine the peak-to-peak ECAP amplitude in accordance with the teachings of International Patent Publication No. WO2015/074121, the contents of which are incorporated herein by reference. Alternative implementations of the ECAP detector may measure and store an alternative characteristic from the neural response, or may measure and store two or more characteristics from the neural response.

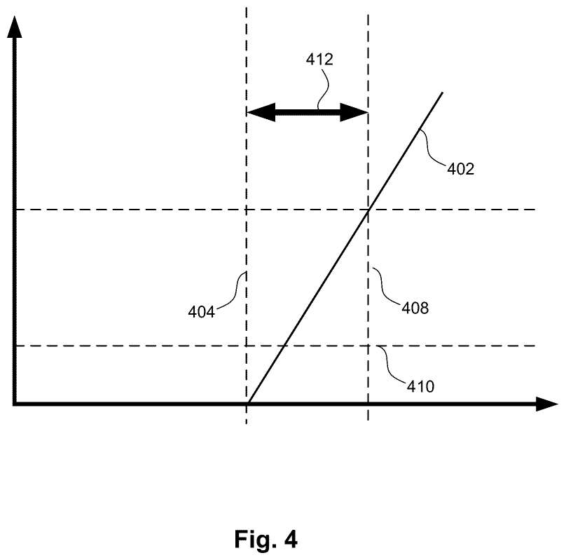

An activation plot, or growth curve, is an approximation to the relationship between stimulus intensity (e.g. an amplitude of the current pulse 160) and intensity of neural response 170 evoked by the stimulus (e.g. an ECAP amplitude). FIG. 4 illustrates an idealised activation plot 402 for one posture of the patient 108. The activation plot 402 shows a linearly increasing ECAP amplitude for stimulus intensity values above a threshold 404 referred to as the ECAP threshold. The ECAP threshold exists because of the binary nature of fibre recruitment: if the field strength is too low, no fibres will be recruited. However, once the field strength exceeds a threshold, fibres begin to be recruited, and their individual evoked action potentials are independent of the strength of the field. The ECAP threshold 404 therefore reflects the field strength at which significant numbers of fibres begin to be recruited, and the increase in response intensity with stimulus intensity above the ECAP threshold reflects increasing numbers of fibres being recruited. Below the ECAP threshold 404, the ECAP amplitude may be taken to be zero. Above the ECAP threshold 404, the activation plot 402 has a positive, approximately constant slope indicating a linear relationship between stimulus intensity and the ECAP amplitude. Such a relationship may be modelled in piecewise linear form as:

$$y = \{ \begin{matrix}S ( s - T ) , & s \geq T \\ 0 , & s < T\end{matrix} \qquad ( 1 )$$

where s is the stimulus intensity, y is the ECAP amplitude, Tis the ECAP threshold and Sis the slope of the activation plot (referred to herein as the sensitivity) above the ECAP threshold T. The sensitivity S and the ECAP threshold T are the key parameters of the activation plot 402.

FIG. 4 also illustrates a discomfort threshold 408, which is a stimulus intensity above which the patient 108 experiences uncomfortable or painful stimulation. FIG. 4 also illustrates a perception threshold 410. The perception threshold 410 corresponds to an ECAP amplitude that is barely perceptible by the patient. There are a number of factors which can influence the position of the perception threshold 410, including the posture of the patient. Perception threshold 410 may correspond to a stimulus intensity that is greater than the ECAP threshold 404, as illustrated in FIG. 4, if patient 108 does not perceive low levels of neural activation. Conversely, the perception threshold 410 may correspond to a stimulus intensity that is less than the ECAP threshold 404, if the patient has a high perception sensitivity to lower levels of neural activation than can be detected in an ECAP, or if the signal-to-noise ratio of the ECAP is low.

For effective and comfortable operation of the neuromodulation device 100, it is desirable to maintain stimulus intensity within a therapeutic range. A stimulus intensity within a therapeutic range 412 is above the ECAP threshold 404 and below the discomfort threshold 408. In principle, it would be straightforward to measure these limits and ensure that stimulus intensity, which may be closely controlled, always falls within the therapeutic range 412. However, the activation plot, and therefore the therapeutic range 412, varies with the posture of the patient 108.

To keep the applied stimulus intensity within the therapeutic range as patient posture varies, in some implementations the neuromodulation device 100 may adjust the applied stimulus intensity based on a feedback variable that is determined from one or more measured ECAP characteristics. In one implementation, the device may adjust the stimulus intensity to maintain the measured ECAP amplitude at a target response intensity. For example, the device may calculate an error between a target ECAP amplitude and a measured ECAP amplitude, and adjust the applied stimulus intensity to reduce the error as much as possible, such as by adding the scaled error to the current stimulus intensity. A neuromodulation device that operates by adjusting the applied stimulus intensity based on a measured ECAP characteristic is said to be operating in closed-loop mode and will also be referred to as a closed-loop neural stimulation (CLNS) device. By adjusting the applied stimulus intensity to maintain the measured ECAP amplitude at or near an appropriate target response intensity, such as a target ECAP amplitude 520 illustrated in FIG. 4b, a CLNS device will generally keep the stimulus intensity within the therapeutic range as patient posture varies.

A CLNS device comprises a stimulator that takes a stimulus intensity value and converts it into a neural stimulus comprising a sequence of electrical pulses according to a predefined stimulation pattern. The stimulation pattern is parametrised by multiple stimulus parameters including stimulus amplitude, pulse width, number of phases, order of phases, number of stimulus electrode poles (two for bipolar, three for tripolar etc.), and stimulus rate or frequency. At least one of the stimulus parameters, for example the stimulus amplitude, is controlled by the feedback loop.

In an example CLNS device, a user sets a target response intensity, and the CLNS device performs proportional-integral-differential (PID) control. In some implementations, the differential contribution is disregarded and the CLNS device uses a first order integrating feedback loop. The stimulator produces a stimulus in accordance with a stimulus intensity parameter, which evokes a neural response in the patient. The intensity of an evoked neural response (e.g. an ECAP) is measured by the CLNS device and compared to the target response intensity.

The measured neural response intensity, and its deviation from the target response intensity, is used by the feedback loop to determine possible adjustments to the stimulus intensity parameter to maintain the neural response at or near the target response intensity. If the target intensity is properly chosen, the patient receives consistently comfortable and therapeutic stimulation through posture changes and other perturbations to the stimulus/response behaviour.

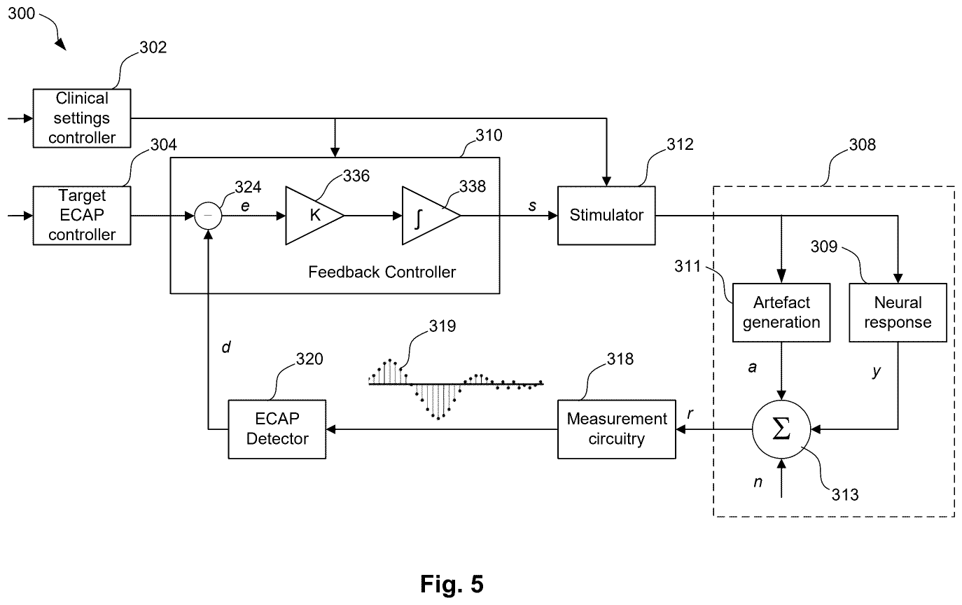

FIG. 5 is a schematic illustrating elements and inputs of a closed-loop neural stimulation (CLNS) device 300, according to one implementation of the present technology. The CLNS device 300 comprises a stimulator 312 which converts a stimulus intensity parameter (for example a stimulus current amplitude) s, in concert with a set of predefined stimulus parameters, to a neural stimulus comprising a sequence of electrical pulses on the stimulus electrodes (not shown in FIG. 5). According to one implementation, the predefined stimulus parameters comprise the number and order of phases, the number of stimulus electrode poles, the pulse width, and the stimulus rate or frequency.

The generated stimulus crosses from the electrodes to the spinal cord, which is represented in FIG. 5 by the dashed box 308. The box 309 represents the evocation of a neural response y by the stimulus as described above. The box 311 represents the evocation of an artefact signal a, which is dependent on stimulus intensity and other stimulus parameters, as well as the electrical environment of the measurement electrodes. Various sources of measurement noise n, as well as the artefact a, may add to the evoked response y at the summing element 313 to form the sensed signal r, including: electrical noise from external sources such as 50 Hz mains power: electrical disturbances produced by the body such as neural responses evoked not by the device but by other causes such as peripheral sensory input: EEG; EMG; and electrical noise from measurement circuitry 318.

The neural recruitment arising from the stimulus is affected by mechanical changes, including posture changes, walking, breathing, heartbeat and so on. Mechanical changes may cause impedance changes, or changes in the location and orientation of the nerve fibres relative to the electrode array(s). As described above, the intensity of the evoked response provides a measure of the recruitment of the fibres being stimulated. In general, the more intense the stimulus, the more recruitment and the more intense the evoked response. An evoked response typically has a maximum amplitude in the range of microvolts, whereas the voltage resulting from the stimulus applied to evoke the response is typically several volts.

Measurement circuitry 318, which may be identified with measurement circuitry 128, amplifies the sensed signal r (potentially including evoked neural response, artefact, and measurement noise), and samples the amplified sensed signal r to capture a “signal window” 319 comprising a predetermined number of samples of the amplified sensed signal r. The ECAP detector 320) processes the signal window 319 and outputs a measured neural response intensity d. In one implementation, the neural response intensity comprises a peak-to-peak ECAP amplitude. The measured response intensity d (an example of a feedback variable) is input into the feedback controller 310. The feedback controller 310 comprises a comparator 324 that compares the measured response intensity d to a target ECAP amplitude as set by the target ECAP controller 304 and provides an indication of the difference between the measured response intensity d and the target ECAP amplitude. This difference is the error value, e.

The feedback controller 310 calculates an adjusted stimulus intensity parameter, s, with the aim of maintaining a measured response intensity d equal to the target ECAP amplitude. Accordingly, the feedback controller 310 adjusts the stimulus intensity parameter s to minimise the error value, e. In one implementation, the controller 310 utilises a first order integrating function, using a gain element 336 and an integrator 338, in order to provide suitable adjustment to the stimulus intensity parameter s. According to such an implementation, the current stimulus intensity parameter s may be determined by the feedback controller 310 as

$$s = \int Kedt \qquad ( 2 )$$

where K is the gain of the gain element 336 (the controller gain). This relation may also be represented as

$$δ s = Ke \qquad ( 3 )$$

where δs is an adjustment to the current stimulus intensity parameter s.

A target ECAP amplitude is input to the feedback controller 310 via the target ECAP controller 304. In one implementation, the target ECAP controller 304 provides an indication of a specific target ECAP amplitude. In another implementation, the target ECAP controller 304 provides an indication to increase or to decrease the present target ECAP amplitude. The target ECAP controller 304 may comprise an input into the CLNS device 300, via which the user can input a target ECAP amplitude, or indication thereof. The target ECAP controller 304 may comprise memory in which the target ECAP amplitude is stored, and from which the target ECAP amplitude is provided to the feedback controller 310.

A clinical settings controller 302 provides clinical settings to the CLNS device 300, including the feedback controller 310 and the stimulus parameters for the stimulator 312 that are not under the control of the feedback controller 310. In one example, the clinical settings controller 302 may be configured to adjust the controller gain K of the feedback controller 310 to adapt the feedback loop to patient sensitivity. The clinical settings controller 302 may comprise an input into the CLNS device 300, via which the user can adjust the clinical settings. The clinical settings controller 302 may comprise memory in which the clinical settings are stored, and are provided to components of the CLNS device 300.

### Multi-AIMD Therapy System

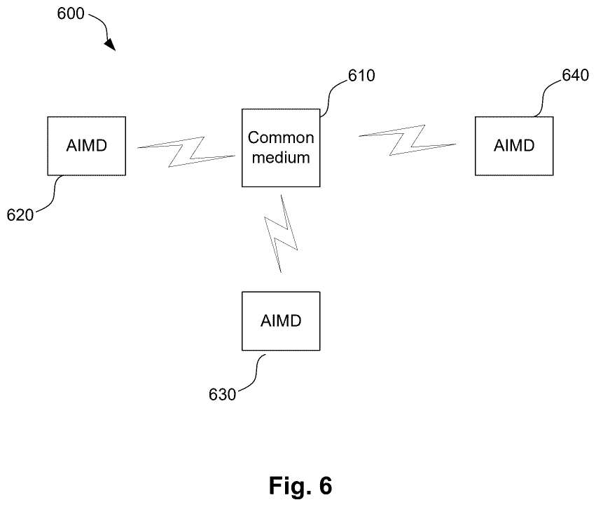

FIG. 6 is a block diagram of a multi-AIMD therapy system 600 according to one implementation of the present technology. The system 600 comprises several AIMDs 620, 630, and 640 implanted within the same patient. The AIMDs 620, 630, and 640 are sufficiently “electrically close” that they may each interact with a common or shared medium 610, through which interference between AIMDs may occur. In one example, the AIMDs are neuromodulation devices positioned to stimulate the same neural tissue (such as the dorsal column) in different locations, and the common medium 610 is the neural tissue. One or more of the AIMDs in the system 600 may be positioned to stimulate peripheral neural tissue, or all the AIMDs may be positioned to stimulate the central nervous system (the spinal cord and the brain).

In another example, one of the AIMDs is an ICD while another AIMD is a CLNS device. In such an example, the common medium 610 is the body tissue generally, since a discharge by the ICD may affect the neural response measurements by a CLNS device. Likewise, a stimulus from a CLNS device may affect the monitoring carried out by an ICD.

In a further example, one of the AIMDs is a deep brain stimulation (DBS) device while another AIMD is a CLNS device. In such an example, the common medium 610 is the body tissue generally, since the crosstalk from a stimulus by the DBS device may affect the neural response measurements by a CLNS device. This is particularly likely if the DBS device stimulates in monopolar fashion. i.e. using the case as a return electrode, because the case of the DBS device is typically implanted at a similar spinal level to the electrode array of a CLNS device through which neural response sensing takes place.

As mentioned above, the activity of each AIMD in a multi-AIMD therapy system such as the system 600 has the potential to interfere with the operation of another AIMD in the system. For example, if one of the AIMDs is a CLNS device that measures ECAPs within a signal window captured after each of its stimuli, and another AIMD is a neuromodulation device that stimulates during that window, there may be interference with the measurement that will affect efficacy of the therapy of the CLNS device. As another example, if two AIMDs in the system are neuromodulation devices that stimulate at the same time, their stimuli may interfere constructively or destructively with one another, affecting the efficacy of the stimuli. In a further example, such interference includes altering, by a first stimulus, the neural response to the second stimulus through mechanisms such as masking. In masking, a nerve is placed in its refractory period and its ability to generate or propagate an action potential is affected. In another example, certain interfaces (most likely in the brain) may sum the effects of the first and second stimuli causing a greater sensation in the patient than either stimulus alone. In yet a further example, a neuromodulation device may capture significant charge from the stimulus of another neuromodulation device at its electrode-tissue interface and DC blocking capacitor, which may be discharged back into the patient in a way that may cause significant discomfort or even injury due to the uncontrolled way in which it is delivered.

In a more extreme case, the activity of one AIMD may damage another AIMD in the system. As one example, if one AIMD in the system is an ICD, its discharge may damage a neuromodulation device in the system if the discharge occurs at a vulnerable time in the device's operation cycle. The general rubric of “interference” is to be interpreted as encompassing such damage for the purposes of the present disclosure.

In some examples of a multi-AIMD therapy system 600, at least one of the devices (an “external” device) may not be implanted. However, interference with its operation may still occur due to the activities of another, implanted device in the system, and vice versa.

Disclosed herein are methods for coordinating the activities of multiple AIMDs within a multi-AIMD therapy system such as the system 600, and multi-AIMD therapy systems configured for such coordination, so as to mitigate any interference between the devices that may damage a device or compromise the efficacy of a device's activity.

According to one aspect of the present technology, the AIMDs are pre-programmed by a “scheduler” entity to operate according to a fixed schedule so as to mitigate any interference. Such programming is carried out before each AIMD goes into operation. In such an implementation, the AIMDs may have independent clocks, but these clocks are synchronised to create shared absolute time between devices, so that all devices are aware of where they are up to in the synchronised schedule at any instant of time.

In one implementation of this aspect in which one AIMD is a neuromodulation device and another AIMD is a CLNS device, the scheduler configures the schedule such that the CLNS device stimulates and measures ECAPs at the proper time to avoid interference from the neuromodulation device's stimuli. In the CLNS device 300 of FIG. 5, the stimuli of the neuromodulation device and those of the CLNS device would be scheduled so that the former do not take place during capture of signal windows 319 of the CLNS device.

In another implementation of this aspect, the system comprises two CLNS devices delivering respective CLNS therapies at different locations in the spine. An ECAP that is evoked at one location will propagate in both directions through the spinal cord and may appear at a delayed time in the other location, potentially affecting the validity of measurements at that location and compromising closed-loop operation. As part of a programming phase, the scheduler instructs a first device to perform a measurement scan in time after the second device evokes an ECAP to identify the delay after which the ECAP may be measured by the first device's measurement electrodes. This operation could then be repeated in reverse to find the other delay. The scheduler may take the delays into account to ensure no interference occurs in either direction. If the relative locations of the two devices are known precisely, the delays in each direction could be estimated without any scanning using the conduction velocities of the fibres being stimulated divided by the distance between them.

One advantage of implementations according to this aspect is that no communication between devices is needed to achieve coordination. However, one difficulty is clock drift. As one device's clock drifts past another's, interference may eventually occur between the devices despite the original schedule. Regular synchronisation pulses from one designated “timer” AIMD to reset all the others' clocks may suffice to mitigate this drift. The frequency of synchronisation pulses will depend on the amount of drift.

This aspect is suitable for systems comprising AIMDs whose operation is predictable and not reactive to bodily phenomena such as arrhythmias.

According to another aspect of the present technology, one AIMD with sufficient computing power is designated as the “master” device and is configured to control the activity of all the other AIMDs (referred to as “slaves”). The master device dynamically arbitrates any potential interference.

In one implementation of this aspect, each AIMD is located in a hierarchy of priorities, e.g. ICDs are assigned a higher priority than neuromodulation devices. The devices operate independently until a conflict in timing occurs that could result in interference. To pre-empt (arbitrate) this conflict, the master instructs the lower priority device to suspend its activity for as long as necessary for the higher priority device to complete its activity. Only then may the lower priority device commence its activity. Alternatively, the lower priority device could be instructed to enter a more robust mode of activity, such as a CLNS device entering open-loop mode. If the lower priority device could be damaged by the activity of the higher priority device, the master may instruct the lower priority device to take preventive action as well, e.g. disconnect all its electrodes.

Implementations according to this aspect are robust to changes in circumstances of the devices such as drifting clocks and are also suitable for systems comprising AIMDs whose operation is unpredictable, i.e. reactive to bodily phenomena such as arrhythmias. However, the master needs to be able to communicate with the slave devices, whether over the common medium 610 or otherwise. Modes of inter-device communication are further described below.

Other aspects of the present technology relate to peer-to-peer or decentralised methods, in that no single AIMD has a special role such as “master” and there is no “scheduler”. Such aspects may make use of parallel operation concepts such as shared logic conditions for signalling back and forth between AIMDs or forming timing agreements between AIMDs as part of a handshake routine.

In one implementation of a peer-to-peer scheme, one AIMD is “active” at a time, executing its activity, while all others are dormant. “Dormant” may include entering a more robust mode of activity, such as open-loop mode for a CLNS device, as well as wholly inactive. “Dormant” may also include entering a mode that is invulnerable to damage by the activity of other AIMDs, such as having all electrodes disconnected. The active device then “hands off” to the next AIMD in a predetermined order by performing a handshake, causing the next AIMD to become active. After this, the preceding AIMD becomes dormant, and the round continues similarly.

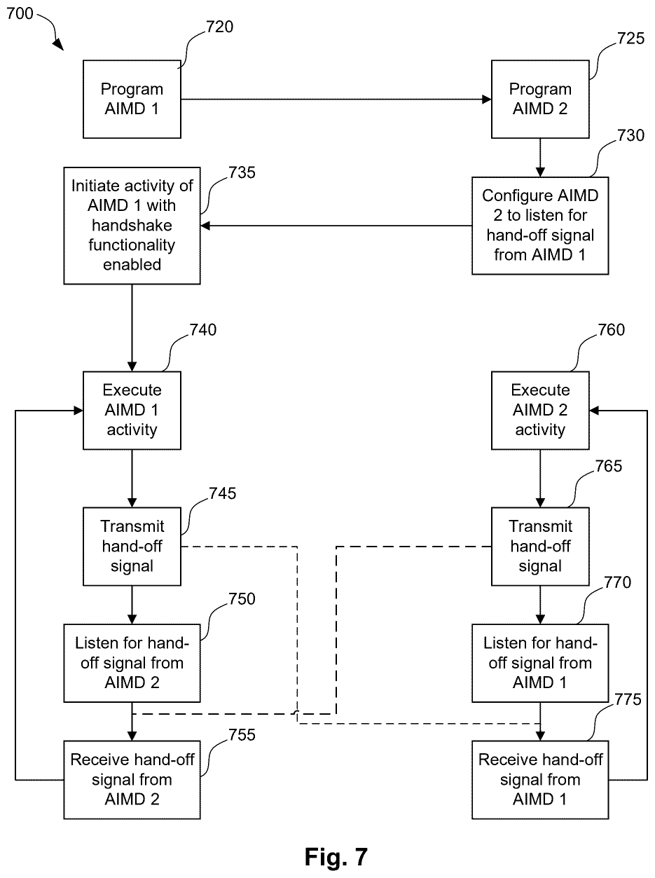

FIG. 7 is a flowchart illustrating a method 700 according to such an implementation of a decentralised coordination scheme. The method 700 is suitable for a multi-AIMD therapy system comprising two AIMDs referred to as AIMDs 1 and 2 (for example, AIMD 620 and AIMD 630 in system 600 of FIG. 6), but may be generalised to a system comprising more than two AIMDs. The method 700 starts at step 720, which programs AIMD 1 in the multi-AIMD therapy system. Step 725 programs AIMD 2 in the multi-AIMD therapy system. Steps 720 and 725 may be implemented by a programming device for the multi-AIMD therapy system such as a clinical interface, and may be executed in any order or simultaneously. Once steps 720 and 725 are completed, step 730 configures AIMD 2 to become dormant, while listening for a hand-off signal from AIMD 1. Step 735 then initiates AIMD 1's activity with handshake functionality enabled. Steps 730 and 735 may be implemented by the programming device, and may be executed in any order or simultaneously. Step 740 executes AIMD 1's activity while AIMD 2 remains dormant. Once AIMD 1's activity is complete, as part of its handshake functionality, at step 745 AIMD 1 transmits a hand-off signal. Step 750 follows, at which AIMD 1 becomes dormant, listening for a hand-off signal from AIMD 2.

Meanwhile, at step 775 AIMD 2 receives the hand-off signal transmitted by AIMD 1 at step 745. The method 700 then proceeds to step 760, at which AIMD 2 executes its activity. Once AIMD 2's activity is complete, at step 765 AIMD 2 transmits a hand-off signal. Step 770 follows, at which AIMD 2 becomes dormant, listening for a hand-off signal from AIMD 1.

At step 755, AIMD 1 receives the hand-off signal transmitted by AIMD 2 at step 765. The method 700 then returns to step 740, at which AIMD 1 executes its activity, and the back-and-forth continues indefinitely. As there is no predetermined schedule of activity, the method 700 is robust to disruptions in the timing of either AIMD such as an unexpectedly long period of activity by either AIMD.

In a variant of the method 700, an AIMD need not wait until the completion of its activity before transmitting the hand-off signal. In such a variant, the hand-off signal may include timing information allowing the receiving device to estimate when it will be free of interference. That way, when the hand-off occurs, the device becoming active knows when interference is likely to occur and can adjust its own activity accordingly. e.g. by adjusting the timing of its activity to execute it in a non-interference window, handing off to the next device without doing anything (on the assumption that the next round will be clearer), or changing its measurement parameters to mitigate the interference. The latter two options are most appropriate for an AIMD that is configured to execute its activity on a strict schedule, such as a neuromodulation device.

The implementations according to the method 700 and its variants require communication between devices for the transmission and reception of hand-off signals, via the common medium 610) or otherwise. Modes of inter-device communication are further described below.

In some implementations of another decentralised aspect, the AIMDs monitor the common medium in the lead-up to their own activity, and self-regulate (e.g. shift) their activity timing if they detect incipient interference (see below). The AIMDs will usually eventually reach a stable, non-interfering configuration of activity timing. This self-regulation can be done by each device autonomously, without need for arbitration by a master device, prior knowledge of other devices” activity in the form of a schedule, or communication between devices.

In another implementation (referred to herein as “synching”) suitable for a system comprising multiple neuromodulation devices, whose patterns of stimulation are typically regular, a device may, by monitoring the common medium over a long period (e.g. 10 seconds), detect other devices' regular periods of activity from regularities of “noise”, and fit its own activity into the pauses between such periods of activity. This removes the need to listen before every activity.

A more complex implementation where synching may be used is in the above-mentioned system comprising two CLNS therapies being delivered by respective AIMDs at different locations in the spine. An ECAP that is evoked at one location will propagate in both directions through the spinal cord and may appear at a delayed time in the other location, potentially affecting the validity of measurements at that location and compromising closed-loop operation. An appropriate activity timing configuration to avoid such interference may be formed by performing a measurement scan in time on one device after the other device evokes an ECAP to identify the delay after which the ECAP may be measured by the first device's measurement electrodes. Using this delay an appropriate timing adjustment could be established by the first device.

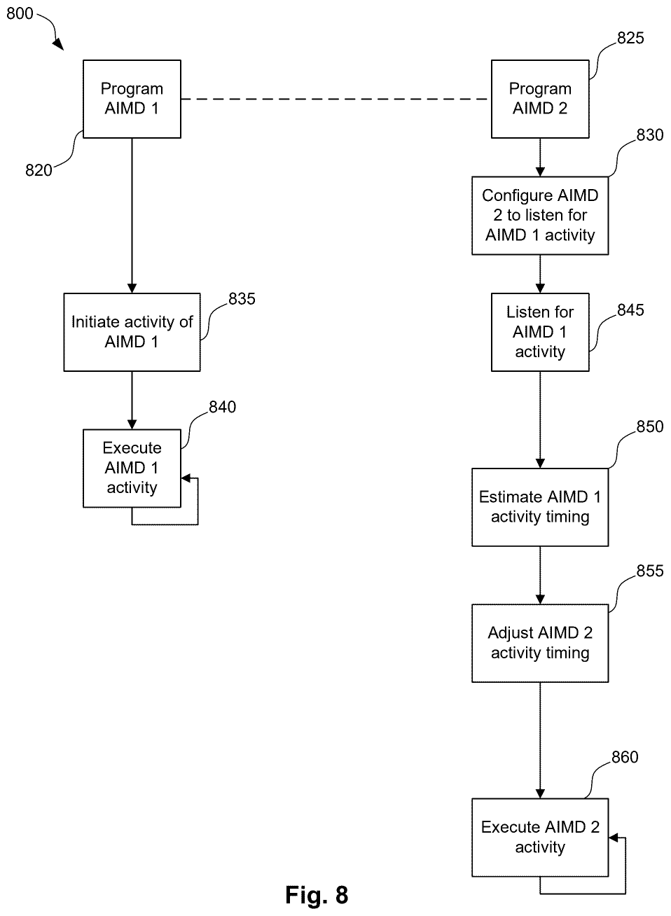

FIG. 8 is a flowchart illustrating a method 800 according to such an implementation. The method 800 is suitable for a multi-AIMD therapy system comprising two AIMDs referred to as AIMDs 1 and 2 (for example, AIMD 620 and AIMD 630 in system 600 of FIG. 6), but may be generalised to a system comprising more than two AIMDs. The method 800 starts at step 820, which programs AIMD 1 in the multi-AIMD therapy system. Step 825 programs AIMD 2 in the multi-AIMD therapy system. Steps 820 and 825 may be implemented by a programming device for the multi-AIMD therapy system such as a clinical interface, and may be executed in any order or simultaneously. Once steps 820 and 825 are completed, step 830 configures AIMD 2 to listen for AIMD 1's activity over the common medium and step 835 initiates AIMD 1's activity. Steps 830 and 835 may be implemented by the programming device, and may be executed in any order or simultaneously. Step 840 executes AIMD 1's activity. Step 840 may be iterated indefinitely by AIMD 1 without reference to AIMD 2. Meanwhile, at step 845 AIMD 2 listens for AIMD 1's activity. Step 845 may continue over many iterations of AIMD 1's activity (step 840). Step 850 then estimates the timing of AIMD 1's activity in relation to its own timing. At the next step 855. AIMD 2 adjusts the timing of its activity so that any interference to its activity by AIMD 1's activity is mitigated. Steps 850 and 855 may be implemented by the programming device. Step 860 then executes AIMD 2's activity according to its adjusted activity timing. Step 860 may be iterated indefinitely by AIMD 2 without reference to AIMD 1.

“Synching” implementations do not require any communication between devices. They also allow for different priorities across the AIMDs. i.e. some AIMDs (e.g. life-preserving devices such as ICDs) have a higher priority than others. A lower priority AIMD detecting interference (e.g. AIMD 2 in FIG. 8) must adjust its timing for a higher priority AIMD (e.g. AIMD 1 in FIG. 8), but a higher priority AIMD need not adjust its timing for a lower priority AIMD whose interference it detects.

An implantable ICD or external life-sustaining device is usually dormant and only discharges rarely and without warning based on its own monitoring of heart rhythms etc., Such a device will be mostly “inaudible” to CLNS devices over the common medium, so the above “synching” implementation will not work for the CLNS devices in a system comprising an ICD. CLNS devices are designed to be discharge-proof to some extent so for such devices there is no significant interference from the occasional discharge (other than a saturated measurement which can be detected and discarded in the usual way). However, a neuromodulation device's stimuli may interfere with the ICD's monitoring, which has potentially serious consequences. The ICD, which as a life-sustaining device is high priority, may use the above-described synching mechanism not to adjust its activity timing, but to adjust its monitoring to distinguish artificial biosignals such as stimuli from irregular heart rhythms and thus avoid false positives.

As mentioned above, some implementations of the present technology require communication of data between AIMDs (e.g. synchronisation pulses to counter clock drift, arbitration, or hand-off signalling). Some examples of communication modes are:
Conductive communication through the nerves
Conductive communication via inter-device wires
Conductive communication wirelessly through the bulk tissue
Radiative communication wirelessly through the tissue (e.g. using a radiofrequency antenna optimised for in-body communication between AIMDs).

Communication may be carried out by modulating or embedding a “communication pattern” encoding data onto a stimulation pattern e.g. amplitude or frequency modulation. Amplitude modulation may be most suitable for high frequency stimulation, e.g. at 100 kHz, while frequency modulation may be more appropriate for stimulation at tonic frequencies.

If one or more of the AIMDs in a multi-AIMD therapy system are external, the above-described coordination methods may work in the same way, provided interference can be detected and communication can take place (if coordination requires it).

In one example, which is suitable for a master/slave implementation, the master device is a smartwatch that has heart-monitoring capability. The smartwatch may detect arrhythmias and inform a neural modulation device to take mitigating action when the smartwatch expects that an ICD might discharge. This prevents the ICD having to inform the neuromodulation device that it is about to discharge.

In another example, the external device is a drug dispenser and the AIMD is a pain-managing CLNS device. If drug intake affects ECAP response, then the CLNS device may react to the dispenser's activity by either increasing or reducing its stimulus intensity to achieve a constant target. One effect of opioids is to increase ECAP amplitude for a given amount of paresthesia, so the CLNS device may reduce its stimulus intensity. Pain relief will be the same, but patient will feel less paresthesia. The patient may then feel the need to increase their target to maintain the same level of paresthesia. To make this unnecessary, the CLNS device may increase its own target when it detects or is informed of action from the drug dispenser.

In another example, the external device is a closed-loop peripheral stimulator such as a TENS device configured to measure EMGs as feedback variables. TENS stimuli may interfere with ECAP measurements by a CLNS device, so the above coordination methods may be employed. Interference between AIMD stimuli and TENS device measurement is less likely although in a case such as sacral nerve stimulation, there can be creation of additional EMGs due to the stimuli which may interfere with TENS measurement of rhythmic EMG. This could occur in the case of patients with diabetic neuropathies which may involve lower limb spasticity or tremors and abdominal nerve disorders (urge incontinence), for which sacral nerve stimulation has been prescribed.

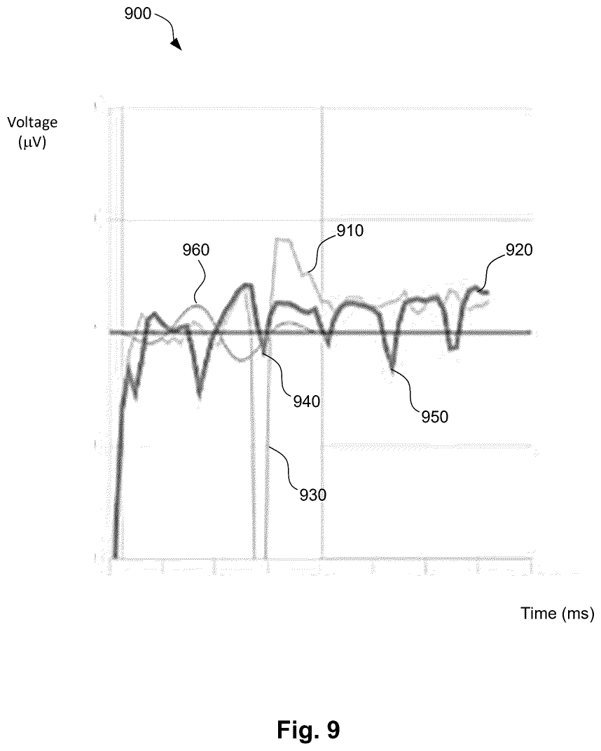

Specific implementations of the “synching” aspect of the present technology are suitable for a multi-AIMD system comprising a DBS device and a CLNS device. As mentioned above, the crosstalk from a stimulus by the DBS device may affect the neural response measurements by the CLNS device, particularly if the DBS device is stimulating in monopolar fashion. This is illustrated in the plot 900 in FIG. 9. The plot 900 contains a thin trace 910 representing a signal window captured by a CLNS device. The trace 910 contains a large negative spike 930 that may be attributed to crosstalk from a stimulus delivered by a co-implanted DBS device. The thick trace 920 represents the average of the last 16 signal windows. It may be seen that the spike 930 has resulted in a smaller but distinct negative spike 940 at the same location in the average trace 920. Other spikes in the average trace 920. e.g. the spike 950, resulting from other large DBS-induced spikes in previous signal windows are also apparent, distributed along the average trace 920. DBS-induced spikes such as the spike 930 tend to corrupt the measurement of ECAP amplitude by the ECAP detector (the trace 960 represents a template that is correlated with the signal window 910 to measure the ECAP amplitude). One example of such corruption is a systematic bias in the ECAP amplitude measurements by the CLNS device. Another example is a significant increase in noise which affects the performance of the feedback loop. This effect is more pronounced if the DBS device stimuli are synchronized and in phase with the stimuli of the CLNS device such that the crosstalk spikes appear repeatedly at or near the same location in the signal window. If on the other hand the stimuli of the CLNS device are synchronized, but out of phase, with the stimuli of the DBS device such that the crosstalk spikes never appear in the signal window (which occurs at a fixed delay after each stimulus), the interference with CLNS device ECAP measurement by crosstalk from the stimuli of the DBS device will be mitigated. Alternatively, the stimuli of the CLNS device may be desynchronised from the stimuli of the DBS device, such that the crosstalk spikes are more evenly distributed along the signal window as evidenced in FIG. 9. This will also mitigate the interference. Both of these mitigations may be classified under the broad heading of “synching”.

One specific implementation of the synching method 800 is configured to achieve desynchronisation. In such an implementation, the DBS device is AIMD 1 and the CLNS device is AIMD 2. The implementation does not require that AIMD 2 has any prior knowledge of how AIMD 1 has been programmed in step 820. According to the implementation, step 830 configures AIMD 2 to deliver its stimuli at a stimulus frequency which gradually varies over a predetermined range. The variation may be random or may be monotonic increasing or decreasing. The stimuli may be sub-threshold so that they do not evoke any ECAPs, and thus any spikes in the signal window may be attributed to crosstalk from AIMD 1. In step 845. AIMD 2 delivers its stimuli at the variable stimulus frequency and captures signal windows in open-loop mode. i.e. without making any adjustments to stimulus intensity based on response intensity. Step 855 adjusts a stimulus frequency for AIMD 2 to be the smallest frequency within the predetermined range of stimulus frequencies for AIMD 2 such that the least common multiple of the inter-stimulus intervals of AIMD 1 and AIMD 2 is greater than or equal to N times the inter-stimulus interval of AIMD 2, where N is an integer such as 20. (The inter-stimulus interval of an AIMD is the reciprocal of the stimulus frequency of the AIMD.) Such a condition will ensure that crosstalk spikes from AIMD 1 do not recur in the same location in the signal window of AIMD 2 more than once in every N signal windows of AIMD 2.

At step 860, AIMD 2 operates normally at the stimulus frequency adjusted at step 855.

In an alternative implementation of the synching method 800 that is configured to achieve synchronisation, steps 820 to 850 and 860 are as just described. However, step 855 adjusts the stimulus frequency of AIMD 2 to be the same as the stimulus frequency of AIMD 1 estimated at step 850, and adjusts a delay between the stimuli of AIMD 1 and the stimuli of AIMD 2 such that the crosstalk spikes from AIMD 1 do not fall within the signal windows of AIMD 2.

The method 800 may be executed once only. Alternatively, the steps 830 to 860 of the method 800 may be repeated, either periodically or when triggered by some event. Repeated execution of steps 830 to 860 is suitable for a multi-AIMD therapy system in which the timing of AIMD 1's activity may be expected to change over time, for example as a result of clock drift. Repeated execution of steps 830 to 860 allows AIMD 2 to adjust its timing to any variation in timing of AIMD 1. In such repeated implementations, steps 845 to 855 may be carried out by AIMD 2.

According to other synching implementations, AIMD 2 may be configured to continually adjust its timing to remain desynchronised from AIMD 1's timing. In one such implementation, AIMD 2 delivers its stimuli according to an inter-stimulus interval that varies from stimulus to stimulus, around a central value whose reciprocal is the desired stimulus frequency of AIMD 2. The variation may be predetermined (e.g. sinusoidal with a predetermined amplitude and frequency) or random. In such an implementation, AIMD 2 may, but does not need to, estimate AIMD 1's timing. Optionally, in such an implementation, AIMD 2 may determine, as part of its feedback loop, a single-ECAP quality measure for each signal window, to determine whether each signal window is likely to have been corrupted by a crosstalk spike. Examples of the determination of a single-ECAP quality measure from a signal window are disclosed in International Patent Publication no. WO2023/115157, the entire contents of which are herein incorporated by reference. If the single-ECAP quality measure falls below a threshold, indicating probable corruption, the signal window may be discarded, i.e. not used to measure an ECAP amplitude or adjust the stimulus intensity. Such supervision of the feedback loop using single-ECAP quality determination helps to further mitigate the interference of AIMD 1 with AIMD 2's ECAP amplitude measurement.

In another implementation of continual adjustment, a phase-locked loop (PLL) is used to track variations in AIMD 1's stimulus frequency, and AIMD 2 delivers its own stimuli and captures signal windows either synchronised (and out of phase) with AIMD 1's stimulus frequency, or desynchronised from it.

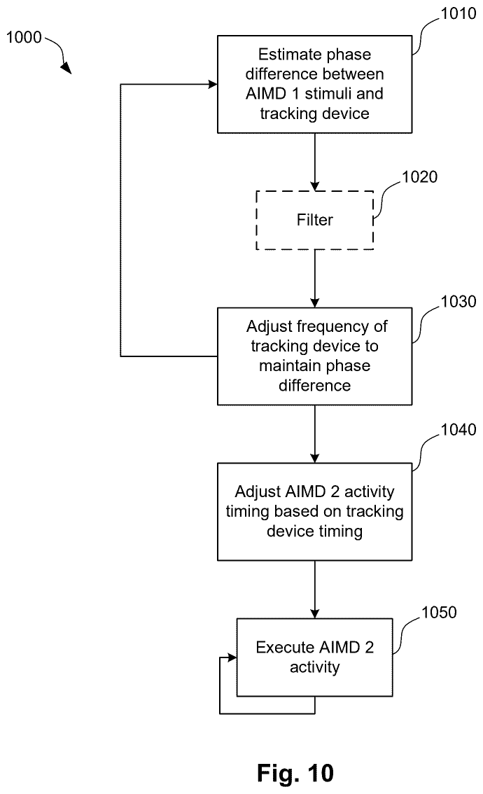

FIG. 10 is a flow chart illustrating a method 1000 according to such an implementation. The method 1000 may be executed after steps 820, 825, 835, and 840 of the method 800 have been carried out to program each AIMD, and AIMD 1's activity is under way. The method 1000 involves a tracking device configured to detect crosstalk spikes indicative of stimuli from AIMD 1 within detection windows periodically captured by the tracking device. Step 1010 estimates a phase difference between the windows of the tracking device and the stimuli of AIMD 1 using the timing of the most recently detected crosstalk spike within the current detection window. An optional step 1020 (shown dashed in FIG. 10) filters the phase difference, for example using a moving average (MA) filter that combines the current measurement with previous measurements of the phase difference. Step 1030 then adjusts the frequency of the tracking device so as to maintain the phase difference between AIMD 1's stimuli and the detection windows of the tracking device at or near a predetermined target phase difference. The phase-locked loop comprises the repeated iteration of steps 1010 to 1030. Meanwhile after each iteration of step 1030, step 1040 adjusts the timing of AIMD 2's stimulus/measurement cycle based on the timing of the tracking device's detection cycle as adjusted at step 1030. In one implementation, step 1040 adjusts the stimulus frequency of AIMD 2 to be the same as the frequency of the tracking device, and adjusts a delay between the windows of the tracking device and the stimuli of AIMD 2 such that the crosstalk spikes from AIMD 1 do not fall within the signal windows of AIMD 2. In another implementation, step 1040 adjusts a stimulus frequency for AIMD 2 to be the smallest frequency within the predetermined range of stimulus frequencies for AIMD 2 such that the least common multiple of the inter-stimulus intervals of AIMD 1 (as provided by the tracking device) and AIMD 2 is greater than or equal to N times the inter-stimulus interval of AIMD 2, where Nis an integer such as 20.

At step 1050. AIMD 2 operates normally at the timing adjusted at step 1040.

The tracking device may be implemented as part of AIMD 2 if the hardware exists to support its functionality within AIMD 2. Alternatively, the tracking device may be a separate device within the multi-AIMD therapy system 600, in communication with AIMD 2. The tracking device may be an external computing device such as the external computing device 192. Steps 1010 to 1040 may be carried out by the tracking device. Alternatively, step 1040 may be carried out by AIMD 2.

Phase-locked loops occasionally fail to keep track of the device being tracked. In a variation of the method 1000, the method 1000 may incorporate the determination of single-ECAP quality measure within the feedback loop of AIMD 2 to detect a loss of tracking by the tracking device. In such a variation, if the single-ECAP quality measure falls below a threshold, indicating the probability of corruption, the signal window may be discarded by AIMD 2, i.e. not used to measure an ECAP amplitude. If the recent rate of discards exceeds a threshold, tracking may be deemed lost and the method 1000 re-started to resynchronise the tracking device with AIMD 1.

Other mitigations that may be used by a CLNS device within a multi-AIMD therapy system, either on their own or in conjunction with the method 800 or the method 1000, include:
Using a sensing electrode connected to the CLNS device to sense the interfering spikes in the tissue voltage at a location that is insensitive to neural responses evoked by the stimuli of the CLNS device. The signal from the sensing electrode, after a predetermined scaling, may be subtracted from the captured signal window of the CLNS device before ECAP measurement.
Programming the feedback loop of the CLNS device with a lower controller gain K than would otherwise be set based on the sensitivity to achieve a predetermined loop speed if the CLNS device were not part of a multi-AIMD therapy system. A lower controller gain lowers the susceptibility of the feedback loop to unwanted noise in the ECAP measurements such as would result from the crosstalk spikes from interfering AIMDs.
Programming the CLNS device with a measurement electrode configuration comprising a recording electrode and a reference electrode separated on the electrode array by one or more contacts. This makes the interfering spikes more “common mode” and therefore of lower intensity in the differentially-sensed signal windows of the CLNS device.

The above mitigations, and in particular the methods 800 and 1000 and their variations described above, are not limited to multi-AIMD therapy systems containing two AIMDs, but may be generalised to multi-AIMD therapy systems containing more than two AIMDs. For example, the steps 830 to 860 may be carried out once for each potentially interfering AIMD. Likewise, multiple tracking devices may be incorporated to track the timing variations respective potentially interfering AIMDs and inform the CLNS device so that the CLNS device may adjust its timing to avoid each one.

### INTERPRETATION

The technology disclosed herein may be implemented in hardware (e.g., using digital signal processors, application specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be implemented as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored or executed in a distributed fashion. The present technology is not limited to any particular programming language or operating system.

### Wireless

In the context of the present disclosure, the term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some embodiments they might not. In the context of the present disclosure, the term “wired” and its derivatives may be used to describe circuits, devices, systems, methods, techniques, communications channels, etc., that may communicate data through the use of modulated signals propagating through a conductive medium. The term does not imply that the associated devices are coupled by electrically conductive wires.

Wireless communication standards that can be accommodated include IEEE 802.11 wireless LANs and links, Bluetooth, and wireless Ethernet. The technology disclosed herein may be implemented using devices conforming to other network standards and for other applications, including, for example other WLAN standards and other wireless standards such as MICS.

### Processes

Unless specifically stated otherwise, as apparent from the following discussions, it is appreciated that throughout the specification discussions utilizing terms such as “processing”, “computing”, “comparing”, “estimating”, “calculating”, “determining”, “analysing” or the like, refer to the action or processes of a computer or computing system, or similar electronic computing device, that manipulate or transform data represented as physical, such as electronic, quantities into other data similarly represented as physical quantities, or to otherwise execute a predefined procedure suitable to effect the described actions.

### Processor

In a similar manner, the term “processor” may refer to any device or portion of a device that processes electronic data, e.g., from registers or memory, to transform that electronic data into other electronic data that, e.g., may be stored in registers or memory. A “computer” or a “computing device” or a “computing machine” or a “computing platform” may include one or more processors.

The methods described herein are, in one embodiment, performable by one or more processors that accept computer-readable (also called machine-readable) code containing a set of instructions that when executed by one or more of the processors cause the one or more processors to carry out at least one of the methods described herein. Any processor capable of executing a set of instructions (sequential or otherwise) that specify actions to be taken are included within the meaning of the term “processor”. Thus, one example is a typical processing system that includes one or more processors. The processing system further may include a memory subsystem including main RAM or a static RAM, or ROM.

### Networked or Multiple Processors

In alternative embodiments, the one or more processors operate as respective standalone device(s) or may be connected, e.g., networked to other processor(s), in a networked deployment. The one or more processors may operate in the capacity of a server or a client machine in server-client network environment, or as a peer machine in a peer-to-peer or distributed network environment. The one or more processors may form a web appliance, a network router, switch or bridge, or any machine capable of executing a set of instructions (sequential or otherwise) that specify actions to be taken by that machine.

Note that while some diagram(s) only show(s) a single processor and a single memory that carries the computer-readable code, those in the art will understand that many of the components described above are included, but not explicitly shown or described in order not to obscure the inventive aspect. For example, while only a single machine may be illustrated, the term “machine” shall also be taken to include any collection of machines that individually or jointly execute a set (or multiple sets) of instructions to perform any one or more of the methods discussed herein.

### Additional Implementations

Thus, one implementation of each of the methods described herein is in the form of a computer-readable medium carrying a set of instructions, e.g., a computer program that are for execution on one or more processors. Thus, as will be appreciated by those skilled in the art, aspects of the present technology may be implemented as a method, an apparatus such as a special purpose apparatus, an apparatus such as a data processing system, or a computer-readable medium. The computer-readable medium carries computer-readable code including a set of instructions that when executed on one or more processors cause the processor or processors to implement a method. Accordingly, aspects of the present technology may take the form of a method, an entirely hardware implementation, an entirely software implementation or an implementation combining software and hardware aspects. Furthermore, the present technology may take the form of a carrier medium (e.g., a computer program product) carrying computer-readable program code embodied in the medium.

### Carrier Medium

The software may further be transmitted or received over a network via a network interface device. While the carrier medium is shown in an example embodiment to be a single medium, the term “carrier medium” should be taken to include a single medium or multiple media (e.g., a centralized or distributed database, or associated caches and servers) that store the one or more sets of instructions. A carrier medium may take many forms, including but not limited to, non-volatile media, volatile media, and transmission media.

### Means for Carrying Out a Method or Function

Furthermore, some of the implementations are described herein as a method or combination of elements of a method that can be implemented by a processor of a processor device, computer system, or by other means of carrying out the function. Thus, a processor with the necessary instructions for carrying out such a method or element of a method forms a means for carrying out the method or element of a method. Furthermore, an element described herein of an apparatus is an example of a means for carrying out the function performed by the element.

Those of skill would further appreciate that the various illustrative logical blocks, modules, and algorithm steps described in connection with the implementations disclosed herein may be implemented as electronic hardware, computer software running on a special purpose machine that is programmed to carry out the operations described in the present disclosure, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the exemplary implementations.

### Implementations

Reference throughout the present disclosure to “one implementation” or “an implementation” means that a particular feature, structure or characteristic described in connection with the implementation is included in at least one implementation of the present technology. Thus, appearances of the phrases “in one implementation” or “in an implementation” in various places throughout the present disclosure are not necessarily all referring to the same implementation, but may refer to different implementations. Furthermore, the particular features, structures or characteristics may be combined in any suitable manner, as would be apparent to one of ordinary skill in the art from this disclosure, in one or more implementations.

Similarly, it should be appreciated that in the above description of example implementations of the present technology, various features are sometimes grouped together in a single implementation, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects may lie in less than all features of a single foregoing disclosed implementation. Thus, the claims following the Detailed Description of the Present Technology are hereby expressly incorporated into this Detailed Description of the Present Technology, with each claim standing on its own as a separate implementation of the present technology.

Furthermore, while some implementations described herein include some, but not other features included in other implementations, combinations of features of different implementations are meant to be within the scope of the present technology, and form different implementations of the present technology, as would be understood by those in the art. For example, in the following claims, any of the claimed implementations can generally be used in any combination.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude or position to indicate that the value or position described is within a reasonable expected range of values or positions. For example, a numeric value may have a value that is +/−0.1% of the stated value (or range of values), +/−1% of the stated value (or range of values), +/−2% of the stated value (or range of values), +/−5% of the stated value (or range of values). +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that each value between two particular values is also disclosed. For example, if 10 and 15 are disclosed, then 11, 12, 13, and 14 are also disclosed.

### Different Instances of Objects

As used herein, unless otherwise specified the use of the ordinal adjectives “first”, “second”, “third”, etc., to describe a common object, merely indicates that different instances of like objects are being referred to, and is not intended to imply that the objects so described must be in a given sequence, either temporally, spatially, in ranking, or in any other manner.

### Specific Details

In the description provided herein, numerous specific details are set forth. However, it is understood that implementations of the present technology may be practiced without these specific details. In other instances, well-known methods, structures and techniques have not been shown in detail in order not to obscure an understanding of the present technology.

### Terminology

Throughout the present disclosure, the terms “a” and “an” mean “one or more”, unless expressly specified otherwise.

Throughout the present disclosure, the word “comprise”, or variations such as “comprises” or “comprising”, will be understood to imply the inclusion of a stated element, integer, or step, or group of elements, integers, or steps, but not the exclusion of any other element, integer, or step, or group of elements, integers, or steps.

Throughout the present disclosure, a statement that an element may be “at least one of” or “one or more of” a list of options is to be understood to mean that the element may be any one of the listed options, or may be any combination of two or more of the listed options.

Throughout the present disclosure, the word “or” is to be read inclusively rather than exclusively, except where otherwise indicated.

Neither the title nor any abstract of the present disclosure should be taken as limiting in any way the scope of the claimed invention.

Where the preamble of a claim recites a purpose, benefit or possible use of the claimed invention, it does not necessarily limit the claimed invention to having only that purpose, benefit or possible use.

In the present specification, terms such as “part”, “component”, “means”, “section”, or “segment” may refer to singular or plural items and are terms intended to refer to a set of properties, functions, or characteristics performed by one or more items having one or more parts. It is envisaged that where a “part”, “component”, “means”, “section”, “segment”, or similar term is described as consisting of a single item, then a functionally equivalent object consisting of multiple items is considered to fall within the scope of the term; and similarly, where a “part”, “component”, “means”, “section”, “segment”, or similar term is described as consisting of multiple items, a functionally equivalent object consisting of a single item is considered to fall within the scope of the term. The intended interpretation of such terms described in this paragraph should apply unless the contrary is expressly stated or the context requires otherwise.

The term “connected” or a similar term, should not be interpreted as being limited to direct connections only. Thus, the scope of the expression “an item A connected to an item B” should not be limited to items or systems wherein an output of item A is directly connected to an input of item B. It means that there exists a path between an output of A and an input of B which may be a path including other items or means. “Connected”, or a similar term, may mean either that two or more elements are in direct physical or causal contact, or that two or more elements are not in direct contact with each other yet still co-operate or interact with each other.

It will be appreciated by persons skilled in the art that numerous variations or modifications may be made to the present technology as shown in the specific implementations without departing from the spirit or scope of the invention as broadly described. For example, any formulas given above are merely representative of procedures that may be used. Functionality may be added or deleted from the block diagrams and operations may be interchanged among functional blocks. Steps may be added or deleted to methods described within the scope of the present technology. The disclosed implementations are, therefore, to be considered in all respects as illustrative and not limiting or restrictive.

The features described in relation to one or more aspects of the present technology are to be understood as applicable to other aspects of the present technology. More generally, combinations of the steps in the method(s) of the present technology or the features of the system(s) or device(s) of the present technology described elsewhere in the present disclosure, including in the claims, are to be understood as falling within the scope of the disclosure of the present disclosure.

### INDUSTRIAL APPLICABILITY

It is apparent from the above that the arrangements described are applicable to the health care industries.

### EXAMPLES OF THE INVENTION

Example 1. A multi-active-implantable-medical-device (AIMD) therapy system comprising:
a closed-loop neural stimulation (CLNS) device configured to execute a first activity comprising periodically delivering neural stimuli and adjusting one or more stimulus parameters so as to maintain a measured neural response intensity at a target value; and
a second active medical device configured to execute a second activity,
wherein a first device in the system is configured to:
execute, upon reception of a first hand-off signal from the second device in the system, its corresponding activity; and
transmit a second hand-off signal to a third device in the system.

Example 2. The system of example 1, wherein the first hand-off signal comprises timing information.

Example 3. The system of example 2, wherein the first device is configured to adjust the execution of the corresponding activity based on the timing information.

Example 4. The system of example 3, wherein the adjustment comprises adjusting the timing of the execution of the corresponding activity so as to mitigate interference from an activity of the device from which the hand-off signal was received.

Example 5. The system of example 3, wherein the adjustment comprises adjust the parameters of the execution of the corresponding activity so as to mitigate interference from an activity of the device from which the hand-off signal was received.

Example 6. The system of any one of examples 1 to 4, wherein the first device is further configured to become dormant after transmitting the hand-off signal.

Example 7. A closed-loop neural stimulation device for controllably delivering neural stimuli, the device comprising:
a plurality of electrodes including one or more stimulus electrodes and one or more measurement electrodes;
a stimulus source configured to provide neural stimuli to be delivered via the one or more stimulus electrodes to a neural pathway of a patient in order to evoke neural responses from the neural pathway;
measurement circuitry configured to capture signal windows from signals sensed on the neural pathway by the one or more measurement electrodes subsequent to respective neural stimuli; and
a control unit configured to:
control, upon reception of a first hand-off signal, the stimulus source to provide a neural stimulus according to one or more stimulus parameters;
control the measurement circuitry to capture a signal window subsequent to the neural stimulus;
transmit, upon completion of capturing the signal window, a second hand-off signal;
measure an intensity of an evoked neural response in the captured signal window;
determine a feedback variable from the measured intensity of the evoked neural response; and
adjust, using a feedback controller, the one or more stimulus parameters so as to maintain the feedback variable at a target value.

Example 8. A method of mitigating interference between devices in a multi-active-implantable-medical-device therapy system, wherein the system comprises:
a closed-loop neural stimulation (CLNS) device configured to execute a first activity comprising periodically delivering neural stimuli and adjusting one or more stimulus parameters so as to maintain a measured neural response intensity at a target value; and
a second active medical device configured to execute a second activity,
the method comprising:
executing, by a first device in the system, upon reception of a first hand-off signal from the second device in the system, its corresponding activity; and
transmitting, by the first device, a second hand-off signal to a third device in the system.

LABEL LIST

| | | | |
|---|---|---|---|
| neuromodulation device | 100 | CLNS device | 300 |
| patient | 108 | clinical settings controller | 302 |
| electronics module | 110 | target ECAP controller | 304 |
| battery | 112 | box | 308 |
| telemetry module | 114 | box | 309 |
| controller | 116 | controller | 310 |
| memory | 118 | box | 311 |
| clinical data | 120 | stimulator | 312 |
| clinical settings | 121 | element | 313 |
| control programs | 122 | measurement circuitry | 318 |
| pulse generator | 124 | signal window | 319 |
| electrode selection module | 126 | ECAP detector | 320 |
| measurement circuitry | 128 | comparator | 324 |
| ground | 130 | gain element | 336 |
| array | 150 | integrator | 338 |
| biphasic stimulus pulse | 160 | activation plot | 402 |
| ECAP | 170 | ECAP threshold | 404 |
| nerve | 180 | discomfort threshold | 408 |
| communications channel | 190 | perception threshold | 410 |
| external computing device | 192 | therapeutic range | 412 |
| target ECAP amplitude | 520 | step | 825 |
| multi-AIMD therapy system | 600 | step | 830 |
| common medium | 610 | step | 835 |
| AIMD | 620 | step | 840 |
| AIMD | 630 | step | 845 |
| AIMD | 640 | step | 850 |
| method | 700 | step | 855 |
| step | 710 | step | 860 |
| step | 720 | plot | 900 |
| step | 725 | trace | 910 |
| step | 730 | trace | 920 |
| step | 735 | spike | 930 |
| step | 740 | spike | 940 |
| step | 745 | spike | 950 |
| step | 750 | trace | 960 |
| step | 755 | method | 1000 |
| step | 760 | step | 1010 |
| step | 765 | step | 1020 |
| step | 770 | step | 1030 |
| step | 775 | step | 1040 |
| method | 800 | step | 1050 |
| step | 810 | | |
| step | 820 | | |

1. A multi-active-implantable-medical-device therapy system comprising:
a closed-loop neural stimulation (CLNS) device configured to repeatedly execute a first activity comprising: delivering neural stimuli according to a stimulus parameter, measuring intensities of responses evoked by the stimuli, and adjusting a stimulus parameter value so as to maintain the measured evoked response intensity at or near a target value; and
a second active medical device configured to repeatedly execute a second activity; and
a tracking device in communication with the CLNS device, the tracking device being configured to monitor a shared medium of the system to estimate the timing of the second activity;
wherein the CLNS device is configured to:
adjust, based on the timing of the second activity, the timing of the first activity such that interference with the first activity by the second activity is mitigated; and
execute the first activity according to the adjusted timing.

2. The system of claim 1, wherein adjusting the timing of the first activity comprises synchronising the timing of the first activity with the timing of the second activity.

3. The system of claim 2, wherein synchronising the timing of the first activity comprises adjusting a frequency of the first activity to be equal to a frequency of the second activity.

4. The system of claim 3, wherein synchronising the timing of the first activity further comprises adjusting a delay of the first activity.

5. The system of claim 4, wherein the delay is adjusted such that the first activity does not occur during the measurement of evoked responses by the CLNS device.

6. The system of claim 1, wherein adjusting the timing of the first activity comprises desynchronising the timing of the first activity from the timing of the second activity.

7. The system of claim 6, wherein desynchronising the timing of the first activity from the timing of the second activity comprises adjusting a first frequency of the first activity to be different from a second frequency of the second activity.

8. The system of claim 7, wherein adjusting the first frequency comprises setting the first frequency such that the least common multiple of inter-activity intervals of the second device and the CLNS device is greater than or equal to N times an inter-activity interval of the CLNS device, where N is an integer.

9. The system of claim 7, wherein adjusting the first frequency comprises setting an inter-stimulus interval to vary around a central value.

10. The system of claim 1, wherein the tracking device is configured to continually estimate the timing of the second activity using a phase-locked loop.

11. The system of claim 10, wherein the CLNS device is further configured to determine a quality measure for each measured response intensity.

12. The system of claim 11, wherein the CLNS device is further configured to, if the quality measure falls below a predetermined threshold, not adjust the stimulus parameter value based on the measured evoked response intensity.

13. The system of claim 1, wherein the tracking device forms part of the CLNS device.

14. A closed-loop neural stimulation device for controllably delivering neural stimuli, the device comprising:
a stimulus source configured to provide neural stimuli to be delivered to a neural pathway of a patient in order to evoke neural responses from the neural pathway;
measurement circuitry configured to capture signal windows from signals sensed on the neural pathway subsequent to respective neural stimuli; and
a control unit configured to repeatedly execute a first activity comprising:
controlling the stimulus source to provide a neural stimulus according to a stimulus parameter;
controlling the measurement circuitry to capture a signal window subsequent to the neural stimulus;
measuring an intensity of an evoked neural response in the captured signal window;
determining a feedback variable from the measured intensity of the evoked neural response; and
adjusting, using a feedback controller, the stimulus parameter so as to maintain the feedback variable at a target value,
wherein the control unit is further configured to:
monitor a medium shared with a second active medical device to estimate the timing of a second activity by the second device;
adjust, based on the timing of the second activity, the timing of the first activity such that interference with the first activity by the second activity is mitigated; and
execute the first activity according to the adjusted timing.

15. A method of mitigating interference between devices in a multi-active-implantable-medical-device therapy system, wherein the system comprises:
a closed-loop neural stimulation (CLNS) device configured to repeatedly execute a first activity comprising delivering neural stimuli according to a stimulus parameter, measuring intensities of responses evoked by the stimuli, and adjusting a stimulus parameter so as to maintain the measured evoked neural response intensity at or near a target value; and
a second active medical device configured to repeatedly execute a second activity, the method comprising:
monitoring a shared medium of the system to estimate the timing of the second activity;
adjusting, based on the timing of the second activity, the timing of the first activity such that interference with the first activity by the second activity is mitigated; and
executing the first activity according to the adjusted timing.

16. The method of claim 15, wherein adjusting the timing of the first activity comprises synchronising the timing of the first activity with the timing of the second activity.

17. The method of claim 16, wherein synchronising the timing of the first activity comprises adjusting a frequency of the first activity to be equal to a frequency of the second activity.

18. The method of claim 17, wherein synchronising the timing of the first activity further comprises adjusting a delay of the first activity.

19. The method of claim 18, wherein the delay is adjusted such that the first activity does not occur during the measurement of evoked responses by the CLNS device.

20. The method of claim 15, wherein adjusting the timing of the first activity comprises desynchronising the timing of the first activity from the timing of the second activity.

21. The method of claim 20, wherein desynchronising the timing of the first activity from the timing of the second activity comprises adjusting a first frequency of the first activity to be different from a second frequency of the second activity.

22. The method of claim 21, wherein adjusting the first frequency comprises setting the first frequency such that the least common multiple of inter-activity intervals of the second device and the CLNS device is greater than or equal to N times an inter-activity interval of the CLNS device, where N is an integer.

23. The method of claim 21, wherein adjusting the first frequency comprises setting an inter-stimulus interval to vary around a central value.

24. The method of claim 15, wherein the estimating the timing of the second activity comprises using a phase-locked loop.

25. The method of claim 24, further comprising determining a quality measure for each measured response intensity.

26. The method of claim 25, further comprising, if the quality measure falls below a predetermined threshold, not adjusting the stimulus parameter value based on the measured response intensity.
